(19) United States
(12) Patent Application Publication
(10) Pub. No.: US 2024/0285973 A1
(43) Pub. Date: Aug. 29, 2024

(54) METHOD FOR POSITIONING OF TARGET TREATMENT TISSUE DURING IMAGE-GUIDED PROCESS, METHOD FOR GENERATING ADAPTIVE RADIOTHERAPY TREATMENT PLANNING, ELECTRONIC DEVICE AND STORAGE MEDIUM

(71) Applicant: Our United Corporation, Shaanxi (CN)

(72) Inventors: Hong CHENG, Shaanxi (CN); Bo LI, Shaanxi (CN)

(21) Appl. No.: 18/588,894

(22) Filed: Feb. 27, 2024

(30) Foreign Application Priority Data
Feb. 28, 2023 (CN) 202310182581.4

(51) Int. Cl. A61N 5/10

(52) U.S. Cl. CPC A61N 5/1038; A61N 5/1039

(57) ABSTRACT

A method for positioning of target treatment tissue is provided, which includes: obtaining a simulated localization image from a preliminary treatment plan for a target object, the simulated localization image including a target volume and a reference volume of a target prescription dose corresponding to the target volume, and the reference volume being configured to indicate a contour volume formed by target isodose lines of the target prescription dose corresponding to the target volume on image layers in the simulated localization image; obtaining a first image-guided image of the target object; performing a rigid registration on the simulated localization image with the first image-guided image; and displaying the first image-guided image and the reference volume of the target prescription dose to determine an extent of overlap between the reference volume of the target prescription dose in the first image-guided image and the target treatment tissue.

### CROSS-REFERENCE TO RELATED APPLICATION

This application claims priority to Chinese Patent Application No. 202310182581.4, filed on Feb. 28, 2023, which is incorporated herein by reference in its entirety.

### TECHNICAL FIELD

The present disclosure relates to the technical field of image guidance, in particular to a method for positioning of target treatment tissue during an image-guided process, a method for generating adaptive radiotherapy treatment planning, an electronic device and a storage medium.

### BACKGROUND

With the rapid development of computer and medical imaging technology, the field of radiotherapy treatment has continuously matured. The use of image-guided radiation therapy (IGRT) to precisely guide radiotherapy treatment is increasingly being applied in clinical applications.

### SUMMARY

In an aspect, the present disclosure provides a method for locating a target volume. The method includes: obtaining a simulated localization image of a target object, the simulated localization image including the target volume; obtaining an image-guided image including the target volume; and regarding a reference volume of a target prescription dose in the simulated localization image as a region-of-interest, performing a registration on the region-of-interest with the image-guided image to obtain an offset of the target volume, where the reference volume of the target prescription dose is configured to indicate a contour volume formed by isodose lines of the target prescription dose on image layers in the simulated localization image.

In another aspect, the present disclosure provides a method for positioning of target treatment tissue during an image-guided process. The method includes: obtaining a simulated localization image from a preliminary treatment plan for a target object, the simulated localization image including a target volume and a reference volume of a target prescription dose corresponding to the target volume, and the reference volume being configured to indicate a contour volume formed by target isodose lines of the target prescription dose corresponding to the target volume on image layers in the simulated localization image; obtaining a first image-guided image of the target object; performing a rigid registration on the simulated localization image with the first image-guided image; and displaying the first image-guided image and the reference volume of the target prescription dose to determine an extent of overlap between the reference volume of the target prescription dose in the first image-guided image and the target treatment tissue.

In yet another aspect, the present disclosure provides a method for generating an adaptive radiotherapy treatment plan. The method includes: in a case of determining that an extent of overlap between a reference volume of a target prescription dose in a first image-guided image and target treatment tissue does not satisfy a preset overlap condition, obtaining an instruction to trigger a start of an adaptive radiotherapy treatment process for a target object, the reference volume being configured to indicate a contour volume formed by target isodose lines of the target prescription dose corresponding to the target volume on image layers in the simulated localization image; and generating the adaptive radiotherapy treatment plan for the target object.

In yet another aspect, the present disclosure provides an electronic device (e.g., a computer device). The electronic device includes: one or more processors, and a memory coupled to the one or more processors. The memory has stored computer program instructions (e.g., one or more application programs) that, when executed on the processors, cause the electronic device to execute the method for locating the target volume, or the method for positioning of target treatment tissue during the image-guided process, or the method for generating the adaptive radiotherapy treatment plan as described in any of the above embodiments.

In yet another aspect, the present disclosure provides a non-transitory computer-readable storage medium, which has stored thereon a computer program that, when executed on a computer device, causes the computer device to execute the method for locating the target volume, or the method for positioning of target treatment tissue during the image-guided process, or the method for generating the adaptive radiotherapy treatment plan as described in any of the above embodiments.

In yet another aspect, the present disclosure provides a computer program product. The computer program product includes computer program instructions that, when executed on a computer device (i.e., the electronic device), enable the computer device to perform steps in the method for locating the target volume, or the method for positioning of target treatment tissue during the image-guided process, or the method for generating the adaptive radiotherapy treatment plan as described in any of the above embodiments.

In yet another aspect, the present disclosure provides a computer program. The computer program, when executed on a computer device (i.e., the electronic device), enables the computer device to perform steps in the method for locating the target volume, or the method for the positioning of target treatment tissue during the image-guided process, or the method for generating the adaptive radiotherapy treatment plan as described in any of the above embodiments.

### BRIEF DESCRIPTION OF THE DRAWINGS

In order to describe technical solutions in the present disclosure more clearly, the accompanying drawings to be used in some embodiments of the present disclosure will be introduced briefly. Obviously, the accompanying drawings to be described below are merely drawings of some embodiments of the present disclosure, and a person of ordinary skill in the art can obtain other drawings according to those drawings. In addition, accompanying drawings in the following description may be regarded as schematic diagrams, and are not limitations on an actual size of a product, an actual process of a method and an actual timing of signals involved in the embodiments of the present disclosure.

FIG. 1 is an exemplary flow chart of a method for locating a target volume, in accordance with some embodiments of the present disclosure;

FIG. 2 is another exemplary flow chart of a method for locating a target volume, in accordance with some embodiments of the present disclosure;

FIG. 3 is yet another exemplary flow chart of a method for locating a target volume, in accordance with some embodiments of the present disclosure;

FIG. 4 is yet another exemplary flow chart of a method for locating a target volume, in accordance with some embodiments of the present disclosure;

FIG. 5 is yet another exemplary flow chart of a method for locating a target volume, in accordance with some embodiments of the present disclosure;

FIG. 6 is yet another exemplary flow chart of a method for locating a target volume, in accordance with some embodiments of the present disclosure;

FIG. 7 is yet another exemplary flow chart of a method for locating a target volume, in accordance with some embodiments of the present disclosure;

FIG. 8 is an exemplary block diagram illustrating an electronic device, in accordance with some embodiments of the present disclosure;

FIG. 9 is an exemplary flow chart of a method for positioning of target treatment tissue during an image-guided process, in accordance with some embodiments of the present disclosure;

FIG. 10 is an exemplary diagram showing an overlapping relationship between a first image-guided image and a reference volume of a target prescription dose, in accordance with some embodiments of the present disclosure; and

FIG. 11 is an exemplary flow chart of a method for generating an adaptive radiotherapy treatment plan, in accordance with some embodiments of the present disclosure.

### DETAILED DESCRIPTION

The technical solutions in the embodiments of the present disclosure will be clearly and completely described below with reference to the accompanying drawings in the embodiments of the present disclosure. Obviously, the described embodiments are only some of the embodiments of the present disclosure, rather than all of the embodiments. All other embodiments obtained on the basis of the embodiments of the present disclosure by a person of ordinary skill in the art shall be included in the protection scope of the present disclosure.

The ordinal terms such as “first” and “second” are only used for descriptive purposes, and are not to be understood as indicating or implying relative importance or implicitly specifying the number of technical features indicated. Thus, features defined with “first” or “second” may explicitly or implicitly include one or more of the features. In the description of the present disclosure, the term “a/the plurality of” means two or more unless otherwise specified.

In the present disclosure, the word “exemplary” is used to mean “serving as an example, illustration, or illustration.” Any embodiment described herein as “exemplary” is not necessarily to be construed as preferred or advantageous over other embodiments. The following description is given to enable any person skilled in the art to practice the present disclosure. In the following description, details are set forth for the purpose of explanation. It will be understood that one of ordinary skill in the art will recognize that the present disclosure may be practiced without these specific details. In other implementations, well-known structures and processes have not been described in detail to avoid obscuring the description of the present disclosure with unnecessary detail. Thus, the present disclosure is not intended to be limited to the embodiments shown but is to be accorded the widest scope consistent with the principles and features disclosed herein.

Unless the context requires otherwise, throughout the description and the claims, the term “comprise” and other forms thereof such as the third-person singular form “comprises” and the present participle form “comprising” are construed as an open and inclusive meaning, i.e., “including, but not limited to”.

In addition, the phrase “according to” or “based on” used herein is meant to be open and inclusive, since a process, step, calculation, or other action that is “according to” or “based on” one or more of the stated conditions or values may, in practice, be based on additional conditions or values exceeding those stated.

It will be noted that methods provided in embodiments of the present disclosure may be performed on electronic devices (such as computer devices), and processing objects of each computer device all exist in the form of data or information. For example, if an object to be processed is time, its essence is time information. It can be understood that if concepts such as size, quantity, position, etc. are mentioned in subsequent embodiments, these concepts all exist in the form of corresponding data for processing by the computer device, which will not be described specifically here.

Embodiments of the present disclosure provide a method for locating a target volume, an electronic device, and a storage medium, which will be respectively described in detail below.

If a patient is diagnosed with a tumor and requires a radiotherapy treatment, since the radiotherapy treatment is performed by a radiotherapy treatment system, a doctor needs to first obtain an image of the patient, such as an image of the patient's diseased region, then develops a treatment planning suitable for the patient according to the tumor of the patient and tissue and organs around the tumor. The treatment planning includes an irradiation volume and irradiation dose of the tumor, an irradiation dose of the tissue and organs around the tumor, and the like. The treatment planning is generally completed on a treatment planning system (TPS) software, and the TPS software generates the treatment planning and sends it to a treatment delivery system and a real-time control unit (RCU), which controls setup parameters of the radiotherapy treatment system, such as a delivery time, and a gantry speed, so as to complete the radiotherapy treatment process.

The radiotherapy treatment for tumors generally includes processes as follows.

Perform a simulated localization on a patient to obtain a simulated localization image of the patient.

For example, an image of the patient obtained by the doctor is generally a computed tomography (CT) image or a magnetic resonance (MR) image. For example, a simulated localization CT is used to obtain a CT image set of the patient by simulatively locating. Considering an example in which the patient is diagnosed with disease of the prostate, a big bore CT is used to perform a simulated localization on the patient; and during a process of obtaining the CT image set of the patient by simulatively located, the patient is in the Head First-Supine (HFS) position, emptying the rectum and keeping the bladder full, and then at least one (e.g., three) metal markers is affixed on the skin surface of the patient's lower abdominal region to mark the central position of the prostate on isocentric slices.

Contour a target volume on the simulated localization image.

The doctor contours the target volume and related normal organs on the simulated localization CT image set. Here, contouring the target volume includes contouring a gross tumor volume (GTV), a clinical target volume (CTV), an internal target volume (ITV), a planning target volume (PTV), a treatment volume (TV) and an irradiation volume (IV). Still considering the example in which the patient is diagnosed with disease of the prostate, the CTV only includes the prostate generally, and the PTV may be obtained by extending the CTV by 5 mm to the patient's back evenly and extending the CTV by 8 mm towards other directions.

Develop and generate a treatment planning using the simulated localization image.

The doctor sets dose information of the tumor and surrounding tissue in the treatment planning system (TPS), and then the TPS generates a treatment planning. In some embodiments of the present disclosure, a treatment planning of a volumetric modulated arc therapy (VMAT) is generated according to the above-mentioned contoured region and using the following exemplary dose limiting condition:
D95: a dose absorbed by 95% of the PTV;
Dmax: the maximum dose absorbed by 1% of the PTV;
Dmin: the minimum dose absorbed by 99% of the PTV;
V65: a percentage of a volume that absorbs a dose greater than or equal to 65Gy (Gray); and
V40: a percentage of a volume that absorbs a dose greater than or equal to 40Gy.

Perform an image guidance on the patient for treatment.

Here, an image guidance is a process of obtaining an image of a patient using various image guidance devices before, during, or after radiation treatment of the patient to locate a tumor, normal tissue and organs, or a contour of the patient's body surface, in order to make radiation adjustments according to changes in their positions, so as to achieve the purpose of precise radiotherapy treatment of the tumor target volume and reduction of irradiation dose to normal tissue.

The purpose of setting up the patient prior to radiotherapy treatment is to move the top of a table on which the patient is lying to cause the patient to be in a specific position, so that radiation rays from the radiotherapy treatment system can be accurately irradiated into the target volume to complete the radiotherapy treatment.

The radiotherapy treatment system may be an accelerator, a Gamma knife, a Cyber knife, or a Tomo radiotherapy treatment system. The image guidance device may be integrated into the radiotherapy treatment system or may be provided as a stand-alone device. The image guidance device may be an MR guidance device, a biorthogonal image guidance device, a cone beam computed tomography (CBCT) image guidance device, an on-rail CT, or an ultrasound guidance device. The present disclosure does not specifically limit types of the radiotherapy treatment system and the image guidance device.

In a method for locating a target volume provided in embodiments of the present disclosure, after the planning is determined, an isodose surface of a target prescription dose is stored as a reference structure in a radiotherapy treatment (RT) structure file where a planned contour resides, and a specific identifier (a reference volume of the target prescription dose) is given. In a case of performing an image guidance on the patient, a simulated localization image of a target object and an image-guided image including the target volume are obtained first, and then regarding the reference volume of the target prescription dose in the simulated localization image as a region-of-interest (ROI), a registration is performed on the ROI with the image-guided image to obtain an offset of the target volume.

Herein, the term “isodose surface” refers to a volume 100% covered by isodose lines. The RT structure file is a digital imaging and communications in medicine (DICOM) file required to be imported into the radiotherapy treatment system, that is, a file with a digital image format in which the composition of each structural region, a corresponding raw DICOM sequence identifier, and partial information of the patient are stored.

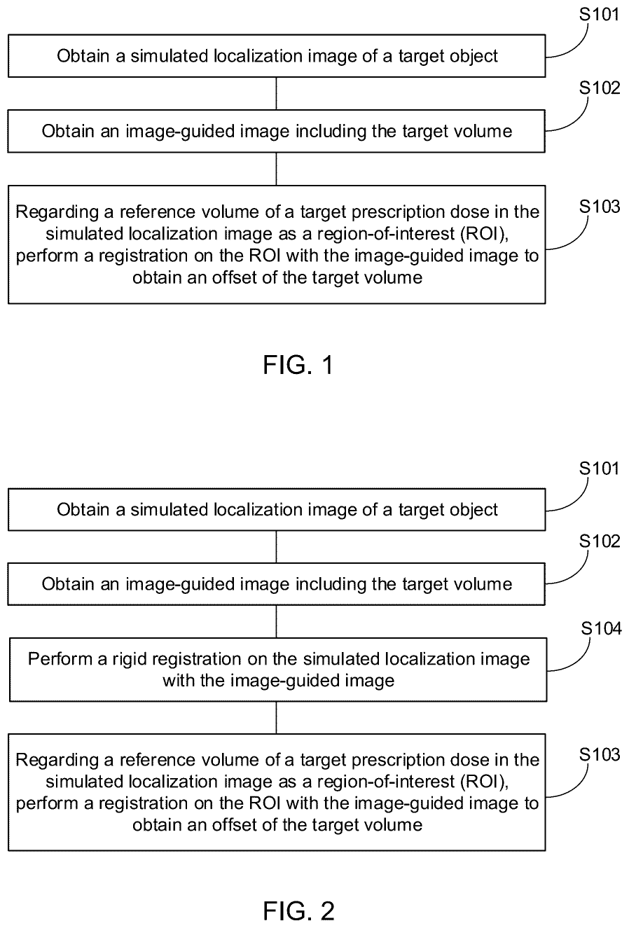

FIG. 1 is an exemplary flow chart of a method for locating a target volume, in accordance with some embodiments of the present disclosure. For example, an execution subject for the method is a processor in a computer device. As shown in FIG. 1, the method includes steps S101 to S103 as follows.

In S101, a simulated localization image of a target object is obtained.

The computer device obtains the simulated localization image of the target object, and the simulated localization image includes the target volume.

In some embodiments, the target object may be a patient (e.g., a diseased region of the patient) or a Phantom.

In some embodiments, the simulated localization image may be a CT image, an MR image, or other images.

Here, the target volume refers to a volume of the target object to be irradiated by rays. It will be noted that the target volume includes at least a gross tumor volume (GTV) of the target object, and of course, may further include other tissue. For example, the target volume may be any one of a gross tumor volume (GTV), a clinical target volume (CTV), a planning target volume (PTV), or other customized volumes.

In S102, an image-guided image including the target volume is obtained.

The computer device may further obtain the image-guided image including the target volume.

In some embodiments, the image-guided image may be a CBCT image, a CT image, an MR image, or other images obtained by the image guidance device. Embodiments of present disclosure do not specifically limit the type of images.

It will be noted that an execution order of S101 and S102 is not limited, that is, S101 and S102 may be performed simultaneously, or S101 is performed before S102, or S101 is performed after S102.

In S103, regarding a reference volume of a target prescription dose in the simulated localization image as a region-of-interest (ROI), a registration is performed on the ROI with the image-guided image to obtain an offset of the target volume.

After obtaining the simulated localization image and the image-guided image, the reference volume of the target prescription dose in the simulated localization image serves as the ROI, and the computer device performs the registration on the ROI with the image-guided image to obtain the offset of the target volume. Here, the reference volume of the target prescription dose is used to indicate a contour volume formed by isodose lines of the target prescription dose on image layers in the simulated localization image. It can also be understood that the reference volume of the target prescription dose is a volume contained in the isodose surface formed by the target prescription dose (i.e., points at which the prescription doses are equal) in the simulated localization image.

In embodiments of the present disclosure, the reference volume of the target prescription dose may be a volume located within the CTV, or may be a volume located within the PTV. The reference volume of the target prescription dose may encompass at least the GTV. For example, the reference volume of the target prescription dose may be a volume located between the GTV and the CTV, or a volume located between the GTV and the PTV, or a volume located between the CTV and the PTV.

It will be noted that the reference volume of the target prescription dose may be a predefined reference volume, or may be automatically generated based on corresponding parameters, or may be manually set by a user (doctor).

In embodiments of the present disclosure, the reference volume of the target prescription dose may be a pre-stored volume, for example, pre-stored in the RT structure file. For example, the reference volume of the prescription dose may be defined in the RT structure file as a file type, and the reference volume of the target prescription dose is pre-stored in a reference volume of a prescription dose in the RT structure file.

It will be further noted that the target prescription dose may be a prescription dose set according to the clinical tumor to be treated, or may be a preset (customized) dose.

For example, in the image-guided radiotherapy treatment (IGRT) for prostate tumors, the anatomical shapes of soft tissue, such as the prostate and prostate tumors, change due to changes in the volume of the rectum and/or bladder, which makes it impossible to accurately match a current image-guided image with the anatomical shape of the prostate in the simulated localization image from the treatment planning, for example, the image-guided image only partially matches the anatomical shape. This may result in the lack of irradiation (under-irradiation) of an objective volume of the prostate tumor and/or a life-threatening over-irradiation of organs adjacent to the prostate.

In light of this, in the method for locating the target volume in embodiments of the present disclosure, after the simulated localization image and the image-guided image are obtained, the reference volume of the target prescription dose in the simulated localization image serves as the region-of-interest (ROI), and the registration is performed on the ROI with the image-guided image to obtain the offset of the target volume. Since the reference volume of the target prescription dose is the reference volume corresponding to the target prescription dose in the treatment planning, the position of the target volume may be accurately located in the image-guided image. In this way, the locating of the target volume may be made accurate, and the possibility of under-irradiation of the target volume and/or over-irradiation of organs at risk (OAR) due to the changes in the anatomical shape of the soft tissue in the target volume is reduced.

In some embodiments, S103 may be performed in different ways as follows to obtain the offset of the target volume.

In a possible implementation, S103 may include: regarding the reference volume of the target prescription dose in the simulated localization image as the region-of-interest (ROI), performing a flexible registration (deformable registration or non-rigid registration) on the ROI with the image-guided image to obtain the offset of the target volume.

For example, the reference volume of the target prescription dose in the simulated localization image may serve as the ROI to perform the flexible registration on the ROI with the image-guided image, based on a deep learning registration model.

In another possible implementation, S103 may include: obtaining an objective volume corresponding to the image-guided image, the objective volume including the target volume; and regarding the reference volume of the target prescription dose in the simulated localization image as the region-of-interest (ROI), performing the flexible registration (deformable registration or non-rigid registration) on the ROI with the objective volume of the image-guided image to obtain the offset of the target volume.

For example, the objective volume corresponding to the image-guided image may be obtained based on a preset deep learning model.

In yet another possible implementation, S103 may include: regarding the reference volume of the target prescription dose in the simulated localization image as the region-of-interest (ROI), adjusting the image-guided image to cause the target volume to be located in the ROI.

Here, the image-guided image can be manually adjusted by the user (e.g., a doctor), or automatically adjusted according to corresponding parameters.

In some embodiments, as shown in FIG. 2, the method may further include a step S104 before S103 as follows.

In S104, a rigid registration is performed on the simulated localization image with the image-guided image.

For example, the rigid registration may be performed on the image-guided image with bony tissue, such as bone, and non-moving soft tissue, in the simulated localization image.

Through S104, the image-guided image can be roughly registered with the simulated localization image, so that the two images are roughly registered.

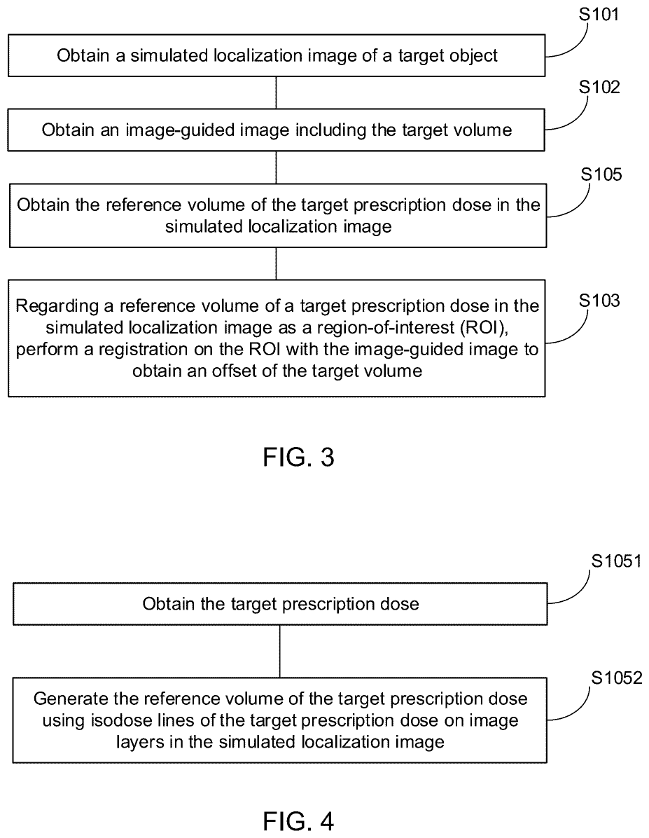

In some embodiments, as shown in FIG. 3, the method may further include a step S105 before S103 as follows.

In S105, the reference volume of the target prescription dose in the simulated localization image is obtained.

It will be noted that there is no order limitation between S105 and both S102 and S103. Embodiments of the present disclosure and the accompanying drawings take S105 between S102 and S103 as an example for illustration.

For example, as shown in FIG. 4, S105 may include sub-steps S1051 and S1052 as follows.

In S1051, the target prescription dose is obtained.

In S1052, the reference volume of the target prescription dose is generated using isodose lines of the target prescription dose on image layers in the simulated localization image.

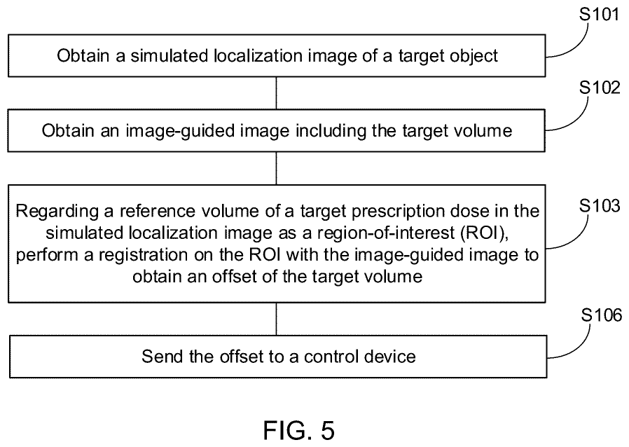

In some embodiments, as shown in FIG. 5, the method may further include a step S106 as follows.

In S106, the offset is sent to a control device to enable the control device to adjust a position of the target object according to the offset.

It will be noted that the control device may be the computer device that performs steps in the above embodiments; alternatively, the control device may be an independent control device, such as a controller, and a position of the top of the table on which the patient is lying is moved through the controller to adjust the position of the target object.

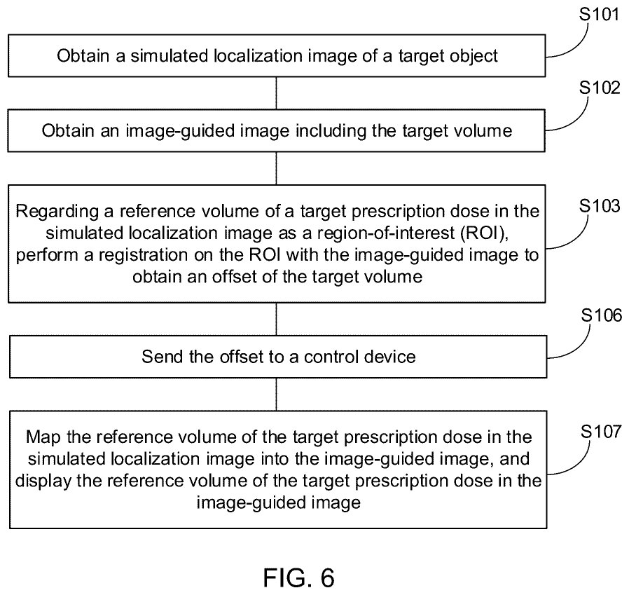

In some embodiments, as shown in FIG. 6, the method may further include a step S107 as follows.

In S107, the reference volume of the target prescription dose in the simulated localization image is mapped into the image-guided image, and the reference volume of the target prescription dose is displayed in the image-guided image.

In this way, the user (such as a doctor) can visually check the position and shape of the reference volume of the target prescription dose in the image-guided image.

It will be noted that S107 is performed after S101 and S102, and there is no sequence between S107 and other steps. For example, S107 may be performed before, after, or simultaneously as any one of S103 to S106, which is not limited in embodiments of the present disclosure, and FIG. 6 only illustrates that S107 is performed after S106 as an example.

Here, the reference volume corresponding to the target prescription dose in the simulated localization image may be set to have a preset color, such as red, blue, green, or other colors, which are not specifically limited in embodiments of the present disclosure.

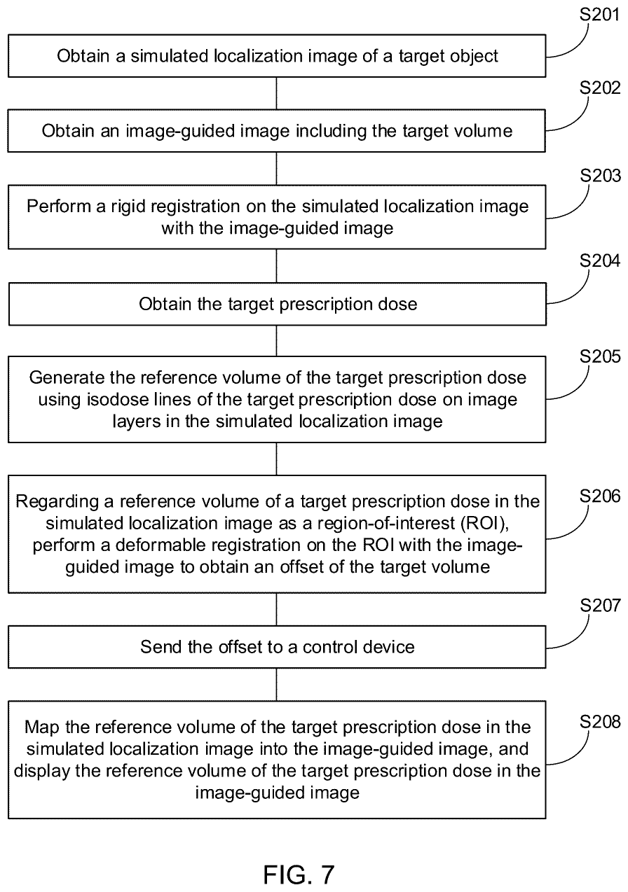

Embodiments of the present disclosure provide another method for locating a target volume. For example, this method is applied to a processor in a computer device. As shown in FIG. 7, the method includes steps S201 to S208 as follows.

In S201, a simulated localization image of a target object is obtained. For the description of S201, reference may be made to the above S101.

In S202, an image-guided image including the target volume is obtained. For the description of S202, reference may be made to the above S102.

In S203, a rigid registration is performed on the simulated localization image with the image-guided image. For the description of S203, reference may be made to the above S104.

For example, before the rigid registration, a rough positioning may be performed according to the metal markers on the patient to obtain a CBCT image set for image guidance (i.e., the image-guided image); a treatment isocenter specified in the treatment planning serves as a center of an image guidance scanning, and the simulated CT image set (i.e., simulated localization image) and the CBCT image set are used for the rigid registration to determine an approximate position of the PTV.

In S204, the target prescription dose is obtained. For the description of S204, reference may be made to the above S1051.

In S205, the reference volume of the target prescription dose is generated using isodose lines of the target prescription dose on image layers in the simulated localization image. For the description of S205, reference may be made to the above S1052.

In S206, regarding a reference volume of a target prescription dose in the simulated localization image as a region-of-interest (ROI), a deformable registration is performed on the ROI with the image-guided image to obtain an offset of the target volume. For the description of S206, reference may be made to the above S103.

For example, a volume contour (i.e., the reference volume of the prescription dose) where the isodose surface of the prescription dose in the treatment planning on the simulated CT image is located serves as the ROI, and the deformable registration based on the ROI is performed to determine a geometric position of the volume encompassed by the isodose surface of the prescription dose in the treatment planning in the CBCT, so as to obtain the offset, where the offset may be a three-dimensional offset (along longitudinal, lateral and vertical directions, which are usually called X, Y, and Z directions respectively), or a six-dimensional offset (along X, Y, and Z linear directions and rotation directions around X, Y, and Z directions).

In S207, the offset is sent to a control device to enable the control device to adjust a position of the target object according to the offset. For the description of S207, reference may be made to the above S106.

For example, a position of the treatment isocenter (i.e., the position of the target object) is moved according to the offset, so that the isodose surface of the prescription dose can encompass a current treatment volume of the prostate target volume of the patient as much as possible. After adjusting the treatment isocenter, the treatment may proceed as planning requirements.

For example, the above method may further include a step after S207 as follows: collecting a post-treatment CBCT image set immediately after the treatment ends, importing the CBCT image set into the TPS, and performing a reconstruction and evaluation of doses to an organ in the target volume and organs at risk (OAR).

In S208, the reference volume of the target prescription dose in the simulated localization image is mapped into the image-guided image, and the reference volume of the target prescription dose is displayed in the image-guided image. For the description of S208, reference may be made to the above S107.

In S201 to S208, the rigid registration (the first registration) is performed on the simulated localization image and the image-guided image, and then the reference volume of the target prescription dose in the simulated localization image serves as the region-of-interest (ROI) based on the rigid registration, a deformable registration is performed on the ROI with the image-guided image. In this way, the locating of the target volume may be made accurate, and the possibility of under-irradiation of the target volume and/or over-irradiation of organs at risk (OAR) due to changes in the anatomical shape of the soft tissue in the target volume is reduced.

Embodiments of the present disclosure provide a method for positioning of target treatment tissue during an image-guided process, which can clearly show a positional relationship between a reference volume of a target prescription dose and target treatment tissue (e.g., a prostate tumor target volume) during the image-guided process, so as to adjust a position of the target object, thereby reducing the probability of under-irradiation of the target treatment tissue and/or the possibility of life-threatening over-irradiation of organs adjacent to the target treatment tissue. For example, the method is applied to a processor in a computer device, and the computer device may be a computer device installed with image-guided system software (IGS), such as an imaging computer device. As shown in FIG. 9, the method includes steps S301 to S304 as follows.

In S301, a simulated localization image is obtained from a preliminary treatment plan for a target object.

The computer device can obtain the simulated localization image from the preliminary treatment plan for the target object. The simulated localization image includes a target volume and a reference volume of a target prescription dose corresponding to the target volume. The reference volume of the target prescription dose corresponding to the target volume is configured to indicate a contour volume formed by target isodose lines of the target prescription dose corresponding to the target volume on image layers in the simulated localization image. For example, the reference volume of the target prescription dose may be a contour volume formed by 95% to 100% of target prescription dose lines, such as a contour volume formed by 95% of the target prescription dose lines, or a contour volume formed by 100% of the target prescription dose lines.

Here, the target object may be a patient to be treated or a phantom. The preliminary treatment plan is a treatment plan developed for the target object based on the simulated localization image, and the treatment plan includes the simulated localization image; the simulated localization image may be a CT image, an MR image, or other images; the target volume is the target treatment tissue after contouring; and the target prescription dose corresponding to the target volume may be a prescription dose set by the user according to a situation of the target volume of the target object.

In some embodiments, the target volume includes at least the GTV of the target object and, of course, may further includes other tissue. For example, the target volume may be any one of GTV, CTV, or PTV or may be another customized volume.

In S302, a first image-guided image of the target object is obtained.

The computer device can obtain the first image-guided image of the target object is obtained. The first image-guided image includes the target treatment tissue, and the first image-guided image may reflect a current situation of the target treatment tissue within the target object. For example, the first image-guided image may be a CBCT image, a CT image, an MR image, or the like obtained by the image guidance device.

It will be noted that the target treatment tissue may not be marked or contoured in the first image-guided image, but rather recognized by the user or computer device.

In some embodiments, the target treatment tissue may also be automatically recognized and contoured by the computer device for easy viewing by subsequent users.

In some embodiments, the target treatment tissue may be a region corresponding to any one of GTV, CTV, or PTV.

In S303, a rigid registration is performed on the simulated localization image with the first image-guided image.

In S304, the first image-guided image and the reference volume of the target prescription dose are displayed, so as to determine an extent of overlap between the reference volume of the target prescription dose in the first image-guided image and the target treatment tissue.

After the computer device performs the rigid registration on the simulated localization image with the first image-guided image, the first image-guided image and the reference volume of the target prescription dose are displayed in real time on a display device, so that it is convenient for the user to determine, or for the computer device to automatically determine, the extent of overlap between the reference volume of the target prescription dose in the first image-guided image and the target treatment tissue, and then to determine whether, by the extent of overlap, if there is a need to move the position of the target object or to adjust the treatment position.

Here, the extent of overlap between the reference volume of the target prescription dose and the target treatment tissue includes an extent of overlap between the shape (or an outer contour) of the reference volume of the target prescription dose and the shape (or an outer contour) of the target treatment tissue, and an extent of overlap between the size of the reference volume of the target prescription dose and the size of the target treatment tissue.

The overlap between the reference volume of the target prescription dose and the target treatment tissue may include a variety of situations, i.e., a complete overlap between the reference volume of the target prescription dose and the target treatment tissue, part or all of the reference volume of the target prescription dose being within the target treatment tissue, part or all of the target treatment tissue being within the reference volume of the target prescription dose. Here, part of the reference volume of the target prescription dose being within the target treatment tissue, or part of the target treatment tissue being within the reference volume of the target prescription dose means that there is a partial overlap between the reference volume of the target prescription dose and the target treatment tissue.

If the extent of overlap is larger, that is, the extent of overlap satisfies a preset overlap condition, for example, the extent of overlap is greater than or equal to a preset overlap threshold or the extent of overlap is within a preset overlap range, it is indicated that the position of the target treatment tissue has not undergone a large shift or has not undergone a shift. In this case, the computer device sends a rigid registration offset obtained by the rigid registration to a treatment delivery system of the radiotherapy treatment system, so as to enable the treatment delivery system to control a real-time control unit (RCU) of the radiotherapy treatment system to adjust the position of the target object according to the rigid registration offset, and to control the radiotherapy treatment system to deliver the radiation to the target object based on the preliminary treatment plan.

On the contrary, if the extent of overlap is smaller, that is, the extent of overlap does not satisfy the preset overlap condition, for example, the extent of overlap is less than the preset overlap threshold or the extent of overlap is not within the preset overlap range, it is indicated that the target treatment tissue has undergone a large shift and/or the target treatment tissue has undergone a large deformation. In this case, the user may manually adjust, or the computer device may automatically adjust, the position of the first image-guided image or the reference volume of the target prescription dose in order to increase the extent of overlap between a contour corresponding to the reference volume of the target prescription dose and the target treatment tissue in the first image-guided image.

In some embodiments, the computer device may automatically determine the extent of overlap between the reference volume of the target prescription dose and the target treatment tissue. The preset overlap threshold or preset overlap range may be set according to the user's actual experience, and a form used to represent the extent of overlap is not limited to a percentage, but of course may be in other forms such as an integer, a decimal, a fraction, a progress bar, or a pie chart. For example, the preset overlap threshold may be any value in a range of 85% to 100%, such as 85%, 90%, 95%, 98%, or 100%; and the preset overlap range may be a range of 85% to 100%, a range of 90% to 100%, a range of 95% to 100%, or other ranges.

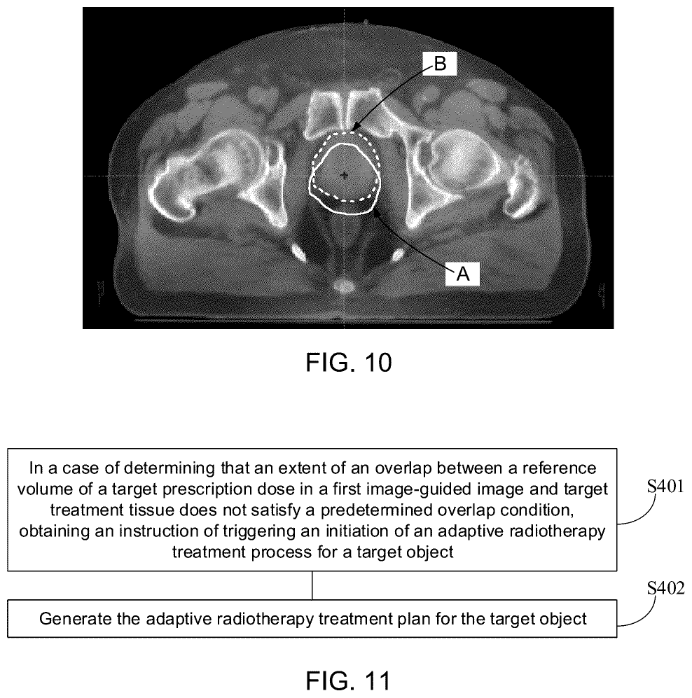

For example, as shown in FIG. 10, after the rigid registration is performed on the first image-guided image with the reference volume, the computer device displays a cross-section of the first image-guided image and the contour corresponding to the reference volume of the corresponding target prescription dose, in which the reference volume is a contour volume formed by 100% prescription dose lines, and the contour corresponding to the reference volume is marked with a solid line A. The cross-section of the first image-guided image shows a cross-section of the internal body tissue of the target object, and the target treatment tissue determined by the user is marked with a dotted line B (which is not displayed in the computer device if it is not automatically contoured), where the target treatment tissue is a region corresponding to the PTV. As can be seen from FIG. 10, the extent of overlap between the reference volume of the target prescription dose and the target treatment tissue is relatively low, which is obviously lower than the preset overlap threshold of 95%, and does not satisfy the preset overlap condition. In this case, the user may manually move the contour corresponding to the reference volume of the target prescription dose or the first image-guided image to increase as much as possible the extent of overlap between the reference volume and the target treatment tissue, so that the target prescription dose of the contour corresponding to the reference volume may be, for example, located within the target treatment tissue in the first image-guided image. It will be noted that in a case where the target treatment tissue is a region corresponding to a CTV or a GTV, the user should try to make the target treatment tissue in the first image-guided image be within the contour corresponding to the reference volume of the target prescription dose.

In embodiments of the present disclosure, the simulated localization image is first obtained from the preliminary treatment plan for the target object, in which the simulated localization image includes the target volume and the reference volume of the target prescription dose corresponding to the target volume, then the first image-guided image of the target object is obtained; after that, the rigid registration is performed on the simulated localization image with the first image-guided image, and the first image-guided image and the reference volume of the target prescription dose are displayed.

Since the reference volume of the target prescription dose corresponding to the target volume can reflect the positional relationship between the target treatment tissue in the preliminary treatment plan and the target volume, and the first image-guided image can reflect a current position of the target treatment tissue, by means of the first image-guided image and the reference volume of the target prescription dose that are displayed, the user can view, or the computer device can automatically estimate, the extent of overlap between the reference volume of the target prescription dose and the target treatment tissue in the first image-guided image to determine whether the current position of the target treatment tissue has undergone a large shift (which may be due to a large deformation of the target treatment tissue). If a large shift occurs, an instruction is given to adjust the position of the target object, so as to reduce the probability of under-irradiation of the target treatment tissue and/or the possibility of life-threatening over-irradiation of organs adjacent to the target treatment tissue.

In some embodiments, S304 may include: based on a result of the rigid registration, mapping the reference volume of the target prescription dose in the simulated localization image into the first image-guided image; and displaying the first image-guided image and the reference volume of the target prescription dose.

In some embodiments, the method may further include, after S304, in a case of determining that the extent of overlap between the reference volume of the target prescription dose in the first image-guided image and the target treatment tissue does not satisfy the preset overlap condition, obtaining an operation of attempting to increase the extent of overlap between the reference volume of the target prescription dose and the target treatment tissue. The operation may be an operation from the user or an operation from the computer device. For example, based on the situation shown in FIG. 10, in a case where it is determined that the reference volume of the target prescription dose in the first image-guided image fails to be completely within the target treatment tissue, a user operation of attempting to place the reference volume of the target prescription dose into the target treatment tissue is obtained.

The attempt by the user to increase the extent of overlap between the reference volume of the target prescription dose and the target prescription tissue results in two outcomes: one possible outcome is that the extent of overlap between the reference volume of the target prescription dose and the target prescription tissue satisfies the preset overlap condition, for example, the extent of overlap is greater than or equal to the preset overlap threshold or the extent of overlap is within the preset overlap range; and the other possible outcome is that the extent of overlap between the reference volume of the target prescription dose and the target prescription tissue still does not satisfy the preset overlap condition, for example, the extent of overlap is less than the preset overlap threshold or the extent of overlap is not within the preset overlap range.

In the first possibility, i.e., it is determined that the extent of overlap between the reference volume of the target prescription dose and the target prescription tissue satisfies the preset overlap condition, a treatment isocenter is correspondingly shifted due to a shift of the target treatment tissue in the first image-guided image. In this case, the computer device sends a treatment isocenter offset indicating the shift that occurs in the treatment isocenter and an offset of the rigid registration to a treatment delivery system of the radiation treatment system, and the treatment delivery system forwards the treatment isocenter offset and the offset of the rigid registration to a treatment planning system (the treatment planning system software may be installable on the electronic device) to cause the treatment planning system to move the treatment isocenter in the preliminary treatment plan accordingly based on the treatment isocenter offset, and to cause the treatment delivery system to control the real-time control unit to deliver radiation to the target object based on the offset of the rigid registration and the preliminary treatment plan with the shifted treatment isocenter.

For example, in a case where it is determined that the reference volume of the target prescription dose is capable of being placed completely within the target treatment tissue, the treatment isocenter offset is sent to the treatment delivery system, and the treatment delivery system forwards the treatment isocenter offset to the treatment planning system (TPS) to cause the treatment planning system to move the treatment isocenter in the preliminary treatment plan accordingly based on the movement offset, and to cause the treatment delivery system to control the real-time control unit to deliver radiation to the target object based on the offset of the rigid registration and the preliminary treatment plan with the shifted treatment isocenter.

In the second possibility, i.e., it is determined that the extent of overlap between the reference volume of the target prescription dose and the target prescription tissue does not satisfy the preset overlap condition, it is indicated that a large deformation has occurred in the target treatment tissue. In this case, the computer device sends an instruction to start an adaptive radiotherapy treatment process directly to the treatment planning system or via the treatment delivery system to the treatment planning system for the treatment planning system to start the adaptive radiotherapy treatment process for the target object.

For example, in a case where it is determined that the reference volume of the target prescription dose fails to be completely in the target treatment tissue, the computer device obtains a user operation for starting the adaptive radiotherapy treatment process and sends an instruction to start the adaptive radiotherapy treatment process to the treatment planning system to cause the treatment planning system to start the adaptive radiotherapy treatment process for the target object. The above steps imply that the user attempts to move the target isodose line into the target volume, but is still unable to place the target isodose line completely into the target treatment tissue, then the adaptive radiotherapy treatment plan is started.

The step involved in the second outcome is a process of starting the adaptive radiotherapy treatment plan. Here, the adaptive radiotherapy treatment plan may refer to a plan that is re-optimized according to the tissue and structure of the target object on the day he/she receives the examination.

The method further includes: after the treatment planning system generates the adaptive radiotherapy treatment plan, re-obtaining an image-guide image, performing a rigid registration on this image-guide image with a planning image for developing the adaptive radiotherapy treatment plan, and then displaying the planning image and the reference volume of the target prescription dose to determine an extent of overlap between the reference volume of the target prescription dose in the planning image and the target treatment tissue. In this way, it is possible to re-verify that the position of the target treatment tissue is accurate, thus ensuring precise delivery of radiation to the target object.

The above steps specifically include: obtaining the planning image for developing the adaptive radiotherapy treatment plan from the adaptive radiotherapy treatment plan for the target object, the planning image including the target volume and the reference volume of the target prescription dose corresponding to the target volume; obtaining a second image-guided image of the target object; performing a rigid registration on the planning image with the second image-guided image; and displaying the planning image and the reference volume of the target prescription dose to determine an extent of overlap between the reference volume of the target prescription dose in the planning image and the target treatment tissue For example, the second image-guided image may be a CBCT image, a CT image, an MR image, or the like obtained by the image guidance device.

For example, the reference volume of the target prescription dose in the planning image is mapped into the second image-guided image to determine whether the reference volume of the target prescription dose in the second image-guided image is capable of being within the target treatment tissue in the second image-guided image.

Embodiments of the present disclosure provide a method for generating an adaptive radiotherapy treatment plan, which may generate the adaptive radiotherapy treatment plan for the target object in a case of determining that an extent of overlap between a reference volume of a target prescription dose corresponding to the target volume of the target object and target treatment tissue does not satisfy the preset overlap condition. For example, the method is applied to a processor in a computer device, and the computer device may be a computer device installed with treatment planning system software, such as treatment planning system computer device. As shown in FIG. 11, the method includes steps S401 and S402 as follows.

In S401, in a case of determining that an extent of overlap between a reference volume of a target prescription dose in a first image-guided image and the target treatment tissue does not satisfy the preset overlap condition, an instruction to trigger a start of the adaptive radiotherapy treatment process for the target object is obtained.

Here, the reference volume is configured to indicate a contour volume formed by target isodose lines of the target prescription dose corresponding to the target volume on image layers in the simulated localization image.

In S402, the adaptive radiotherapy treatment plan for the target object is generated.

It will be noted that the adaptive radiotherapy treatment plan may be a plan obtained by re-optimization based on the first image-guided image or an adaptive radiotherapy treatment plan generated based on the re-obtained simulated localization image.

In some embodiments, the reference volume of the target prescription dose is pre-stored in a radiotherapy treatment (RT) structure file.

Embodiments of the present disclosure provide an electronic device, such as a computer device. The electronic device includes: one or more processors, and a memory coupled to the one or more processors. The memory has stored computer program instructions, e.g., one or more application programs. When executed on the processors, the computer program instructions cause the electronic device to execute the method for locating the target volume, or the method for positioning of target treatment tissue during the image-guided process, or the method for generating the adaptive radiotherapy treatment plan as described in any of the above embodiments.

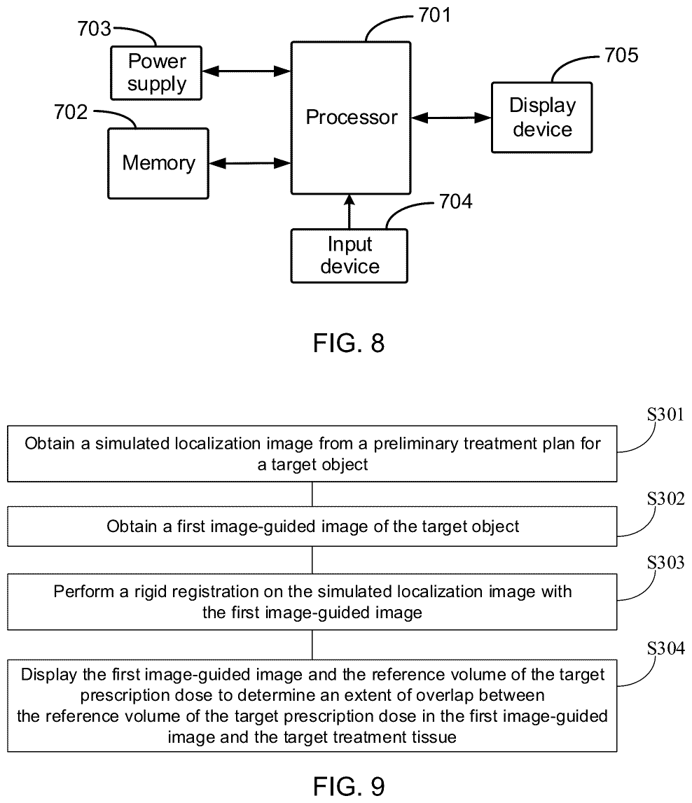

Embodiments of the present disclosure provide an electronic device, such as a computer device. As shown in FIG. 8, the computer device may include one or more processors 701, and one or more memory 702 including a computer-readable storage medium. Those skilled in the art can understand that the composition of the computer device shown in FIG. 8 does not constitute a limitation on the computer device. In some other possible implementations, the computer device may include more or fewer components as shown in the FIG. 8, or may include a combination of some of the components as shown in the FIG. 8, or have a different arrangement of the components than that shown in the FIG. 8.

The processor 701 is the control center of the computer device, which uses various interfaces and lines to connect various parts of the entire computer device. The processor 701 can conduct overall monitoring of the computer device by running or executing software programs and/or modules stored in the memory 702, calling data stored in the memory 702, performing various functions of the computer device and processing the data.

For example, the processor 701 may include one or more processing cores. For example, the processor 701 may integrate an application processor and a modem processor, in which the application processor mainly processes an operating system, a user interface, and an application program, and the modem processor mainly processes wireless communication. It can be understood that the foregoing modem processor may not be integrated into the processor 701.

The memory 702 can be used to store software programs and modules, and the processor 701 executes various functional applications and data processing by executing the software programs and modules stored in the memory 702. The memory 702 may mainly include a storage program area and a storage data area. The storage program area can store the operating system, an application required for at least one function (such as a sound play function and an image play function), etc.; and the storage data area can store data created according to the use of the computer device, etc. In addition, the memory 702 may include a high-speed random access memory, or non-volatile memory, such as at least one magnetic disk storage device, flash memory device, or other non-volatile solid-state storage device. Accordingly, the memory 702 may further include a memory controller to provide the processor 701 with access to the memory 702.

The computer device may further include a power supply 703 for powering various components. For example, the power supply 703 may be logically connected to the processor 701 through a power management system, so as to implement functions such as management of charging, discharging, and power consumption through the power management system. The power supply 703 may include one or more direct current (DC) or alternating current (AC) power supplies, a recharging system, a power failure detection circuit, a power converter or inverter, a power status indicator, and other arbitrary components.

The computer device may further include an input device 704, which can be used to receive input digital or character information, and generate output related to user settings and function control. The input device may be, for example, a keyboard, a mouse, an operating lever, or a touch screen which are capable of receiving input.

Although not shown, the computer device may further include a display device 705 and the like, and the display device 705 may be a display, which will not be described again here. In these embodiments, the processor 701 in the computer device will load executable files corresponding to processes of one or more application programs into the memory 702 according to the following instructions, and the processor 701 will execute the application programs stored in the memory 702, thus realizing various functions, such as the method for locating the target volume, or the method for positioning of target treatment tissue during the image-guided process, or the method for generating the adaptive radiotherapy treatment plan as described in any of the above embodiments.

In some embodiments, the computer device may be a single server, or may be a server network or server cluster composed of multiple servers. For example, the computer device described in the embodiments of present disclosure includes, but is not limited to, a computer, a network host, a single network server, multiple network server sets, or a cloud server composed of multiple servers. Among them, the cloud server consists of a large number of computers or network servers based on cloud computing (CC).

In some embodiments, the aforementioned computer device may be a general-purpose computer device or a special-purpose computer device. In specific implementations, the computer device may be a desktop computer, a portable computer, a network server, a personal digital assistant (PDA), a mobile phone, a tablet computer, a wireless terminal device, a communication device, an embedded device, or the like. These embodiments do not limit the type of computer device.

The computer device may be an imaging computer device, which can perform a communicate connection with a treatment planning system (TPS) computer device, an oncology information management system (OIS), a radiotherapy treatment system treatment delivery system, a radiotherapy treatment system a real-time control unit and other device; alternatively, the computer device may be other computer device, which can perform the communicate connection with the treatment planning system (TPS) computer device, the oncology information management system (OIS), the radiotherapy treatment system treatment delivery system, the radiotherapy treatment system a Real-time control unit and other device.

In the embodiments of the present disclosure, any communication method can be used between the computer device and other devices, including but not limited to, mobile communications based on the 3rd Generation Partnership Project (3GPP), Long Term Evolution (LTE), or Worldwide Interoperability for Microwave Access (WiMAX), or computer network communications based on Transmission Control Protocol/Internet Protocol Suite (TCP/IP Protocol Suite), User Datagram Protocol (UDP), or Digital Imaging and Communications in Medicine (DICOM).

Those of ordinary skill in the art can understand that all or some of the steps in the various methods of the above embodiments can be completed by instructions, or by controlling relevant hardware through instructions. The instructions can be stored in a computer-readable storage medium, and be loaded and executed by the processor.

In light of this, embodiments of the present disclosure provide a non-transitory computer-readable storage medium, which may include: a read-only memory (ROM), a random access memory (RAM), a disk or CD, or the like. A computer program is stored on the non-transitory computer-readable storage medium, and when executed on the computer device (for example, loaded by the processor), the computer program causes the computer device (or processor) to execute the steps in any one of the methods for locating the target volume provided by the embodiments of the present disclosure. For example, the computer program being loaded by the processor may perform the steps in the method for locating the target volume, or the method for positioning of target treatment tissue during the image-guided process, or the method for generating the adaptive radiotherapy treatment plan as described in any of the above embodiments.

In the above-mentioned embodiments, the descriptions of each embodiment have their own emphases. For a part of a certain embodiment that is not described in detail can be found in the detailed description above for other embodiments, and will not be repeated here.

During specific implementation, each of the above structures can be implemented as an independent entity, or can be combined in any way to be implemented as the same or several entities. For the specific implementation of each of the above structures, please refer to the previous method embodiments, and will not be described again here.

For the specific implementation of each of the above operations, please refer to the previous embodiments and will not be described again here.

Embodiments of the present disclosure provide a computer program product. The computer program product includes computer program instructions that, when executed on a computer device (i.e., the electronic device), enable the computer device to perform steps in the method for locating the target volume, or the method for positioning of target treatment tissue during the image-guided process, or the method for generating the adaptive radiotherapy treatment plan as described in any of the above embodiments.

Embodiments of the present disclosure provide a computer program. The computer program, when executed on a computer device (i.e., the electronic device), enables the computer device to perform steps in the method for locating the target volume, or the method for positioning of target treatment tissue during the image-guided process, or the method for generating the adaptive radiotherapy treatment plan as described in any of the above embodiments.

The technical solutions provided by the embodiments of the present disclosure are introduced in detail above. Specific examples are used in this specification to illustrate the principles and implementations of the present disclosure. The description of the above embodiments is only used to help understand the method and its core of the present disclosure. Also, for those skilled in the art, according to the idea of the present disclosure, there will be changes in the specific implementation and application scope. In summary, the content of this specification should not be construed as limiting the present disclosure.

What is claimed is:

1. A method for positioning of target treatment tissue during an image-guided process, comprising:
obtaining a simulated localization image from a preliminary treatment plan for a target object, the simulated localization image including a target volume and a reference volume of a target prescription dose corresponding to the target volume, wherein the reference volume is configured to indicate a contour volume formed by target isodose lines of the target prescription dose corresponding to the target volume on image layers in the simulated localization image;
obtaining a first image-guided image of the target object;
performing a rigid registration on the simulated localization image with the first image-guided image; and
displaying the first image-guided image and the reference volume of the target prescription dose to determine an extent of overlap between the reference volume of the target prescription dose in the first image-guided image and the target treatment tissue.

2. The method of claim 1, wherein displaying the first image-guided image and the reference volume of the target prescription dose, includes:
mapping the reference volume of the target prescription dose in the simulated localization image into the first image-guided image; and
displaying the first image-guided image and the reference volume of the target prescription dose.

3. The method of claim 2, further comprising:
in a case of determining that the extent of overlap between the reference volume of the target prescription dose in the first image-guided image and the target treatment tissue does not satisfy a preset overlap condition, obtaining an operation of attempting to increase the extent of overlap between the reference volume of the target prescription dose and the target treatment tissue.

4. The method of claim 3, further comprising:
in a case of determining that the extent of overlap between the reference volume of the target prescription dose in the first image-guided image and the target treatment tissue still does not satisfy the preset overlap condition, sending an instruction to start an adaptive radiotherapy treatment process to a treatment planning system to cause the treatment planning system to start the adaptive radiotherapy treatment process for the target object.

5. The method of claim 3, further comprising:
obtaining a planning image for developing the adaptive radiotherapy treatment plan from the adaptive radiotherapy treatment plan for the target object, the planning image including the target volume and the reference volume of the target prescription dose corresponding to the target volume;
obtaining a second image-guided image of the target object;
performing a rigid registration on the planning image with the second image-guided image; and
displaying the planning image and the reference volume of the target prescription dose to determine an extent of overlap between the reference volume of the target prescription dose in the planning image and the target treatment tissue.

6. The method of claim 1, wherein the first image-guided image is any one of:
a cone beam computed tomography (CBCT) image, a computed tomography (CT) image and a magnetic resonance (MR) image.

7. The method of claim 1, wherein the simulated localization image is any one of:
a computed tomography (CT) image and a magnetic resonance (MR) image.

8. The method of claim 5, wherein the second image-guided image is any one of:
a cone beam computed tomography (CBCT) image, a computed tomography (CT) image and a magnetic resonance (MR) image.

9. A method for generating an adaptive radiotherapy treatment plan, comprising:
in a case of determining that an extent of overlap between a reference volume of a target prescription dose in a first image-guided image and target treatment tissue does not satisfy a preset overlap condition, obtaining an instruction to trigger a start of an adaptive radiotherapy treatment process for a target object, wherein the reference volume is configured to indicate a contour volume formed by target isodose lines of the target prescription dose corresponding to the target volume on image layers in the simulated localization image; and
generating the adaptive radiotherapy treatment plan for the target object.

10. The method of claim 9, wherein the reference volume of the target prescription dose is pre-stored in a radiotherapy treatment (RT) structure file.

11. An electronic device, comprising:
one or more processors; and
a memory coupled to the one or more processors, wherein
the memory has stored computer program instructions that, when executed on the processors, cause the electronic device to execute steps in the method of claim 1.

12. An electronic device, comprising:
one or more processors; and
a memory coupled to the one or more processors, wherein
the memory has stored computer program instructions that, when executed on the processors, cause the electronic device to execute steps in the method of claim 9.

13. A non-transitory computer-readable storage medium, having stored thereon a computer program that, when executed on a computer device, causes the computer device to execute steps in the method of claim 1.

14. A non-transitory computer-readable storage medium, having stored thereon a computer program that, when executed on a computer device, causes the computer device to execute steps in the method of claim 9.
